(19) United States
(12) Patent Application Publication
(10) Pub. No.: US 2003/0215463 A1
(43) Pub. Date: Nov. 20, 2003

(54) Means of inducing durable immune responses

(76) Inventors: David Knipe, Auburndale, MA (US); Mark Brockman, Boston, MA (US)

Correspondence Address: EDWARDS & ANGELL, LLP, P.O. BOX 9169, BOSTON, MA, 02209, US

(21) Appl. No.: 10/395,822

(22) Filed: Mar. 24, 2003

Related U.S. Application Data
(60) Provisional application No. 60/366,977, filed on Mar. 22, 2002.

(51) Int. Cl. A61K 39/12; A61K 39/245

(52) U.S. Cl. 424/199.1; 424/231.1

(57) ABSTRACT

A replication defective herpes virus vector is described. The replication defective herpes virus vector has a deletion of at least a fragment of a U_{L}29 gene that is replaced with a heterologous sequence encoding an antigen from a specific infectious disease agent. The vector can express said antigen. Prior HSV infection did not diminish the magnitude or the durability of the IgG antibody response generated by preferred replication-defective HSV-1 vectors. A method of inducing in a mammal an immune response against a specific infectious disease agent also is described. A recombinant replication defective mutant Herpes Simplex Virus as a vaccine is administered to a mammal to elicit in the mammal an immune response against the infectious disease causing agent.

The present application claims the benefit of U.S. provisional application No. 60/366,977 filed Mar. 22, 2002, and which is incorporated herein by reference in its entirety.

At least part of the work contained in this application was performed under government grants CA026345 and AI046006 from the National Institutes of Health, U.S. Department of Health and Human Services. The government may have certain rights in this invention.

### FIELD OF THE INVENTION

This invention relates to the construction of vectors that produce a durable protective immune response and may be used repeatedly and still obtain a strong immune response, regardless of prior host immunity.

### BACKGROUND OF THE INVENTION

Herpes Simplex Virus (HSV) infection often results in a localized lesion within epithelial cells of the skin or a mucosal membrane. The innate immune response, consisting of macrophages, natural killer (NK) cells, cytokines, and complement proteins, may act to contain initial viral infection. NK cell-mediated lysis and numerous cytokines, including interleukin (IL)-12, IL-18, and gamma interferon and tumor necrosis factor-alpha, have been reported to affect HSV pathogenesis in mouse models of disease.

The adaptive immune response to infection is comprised of CD8⁺ and CD4⁺ cells, and clearance of viral lesions may involve cytotoxic CD4⁺ T cells. Despite clearance of the virus from peripheral cells, nerves, tissues, etc., HSV can establish a life-long latent infection within the sensory neurons that innervate the site of primary infection. In humans, periodic reactivation of this latent virus results in a recurring disease at or near the site of primary exposure.

Currently, many vector systems are being developed for use in vaccine design. However, a major concern that affects all of these vaccine systems is the potential that prior host immunity may result in diminished efficacy of the vector or threaten the ability to use the same vector for repeated vaccinations.

A need therefore, exists for a vaccine whereby the prior host immunity does not result in diminished efficacy of the vector or threaten the ability to use the same vector for repeated vaccinations.

### SUMMARY OF THE INVENTION

The present invention is directed to a replication defective herpes simplex virus vector wherein the vector induces humoral and cellular immune responses to a specific antigen of choice. In particular the ability of the vector to induce antibody or cellular responses to a specific antigen remains strong even though a subject may have preexisting immunity to the viral vector. In preferred embodiments, the immune responses to the antigen following vector administration to an animal remain extremely durable in both immunized and naïve animals. Thus, in particularly preferred embodiments of the present invention, the ability of a replication-defective HSV-derived vector to elicit long-lived immune responses in animals is not impaired by prior HSV exposure.

In a preferred embodiment, a method of inducing an immune response in a mammal against infectious disease agents comprises: administering to said mammal a sufficient amount of a recombinant replication defective mutant Herpes Simplex Virus comprising a herpes simplex virus wherein the entire U_{L}29 gene or fragments thereof, are deleted and, in the site of the deleted U_{L}29 gene or deleted fragments thereof, a heterologous sequence encoding an antigen from a specific infectious disease causing agent is inserted, which when administered to the mammal in said replication defective mutant herpes simplex virus, elicits an immune response against the infectious disease causing agent. A “deleted fragment” as used herein refers to the percentage of nucleic acids deleted from a gene, for example, the U_{L}29 gene. A deleted fragment comprises at least about 0.1% of contiguous deleted nucleic acids to about 80%, 85%, 90%, or 99.9% contiguously deleted nucleic acids of a gene, such as the U_{L}29 gene.

In accordance with the invention, the inserted heterologous sequence preferably comprises a promoter which directs the expression of DNA encoding an antigen from a specific infectious disease causing agent, or the inserted sequence coding for an antigen from a specific infectious disease causing agent is under the control of the herpes simplex virus promoter.

In another preferred embodiment, the replication defective mutant herpes simplex virus elicits a durable humoral and cellular immune response specific for the antigen encoded by said herpes virus in both naïve and pre-immunized animals.

In other preferred embodiments of the invention, the expression product of the inserted sequence is comprised of one or more antigenic epitopes.

In another preferred embodiment, the replication defective mutant herpes simplex virus can be used in a series of inoculations with at least one other vaccine. The vaccine and the vector can induce the same specific immune responses to the same antigen or each can elicit an immune response to different epitopes of the antigen.

In another preferred embodiment, the replication defective mutant herpes simplex virus is used to increase the durability of immune responses by immunizing a patient with the replication defective mutant herpes simplex virus at least two or more times.

The replication defective mutant herpes simplex virus can be administered in various routes including, for example, subcutaneously, intranasally, intratracheally, or intramuscularly.

In another embodiment of the invention, the vector comprises a replication defective herpes virus vector comprising a deleted U_{L}29 gene or fragments thereof, and inserting into said deleted U_{L}29 gene, a heterologous sequence encoding an antigen from a specific infectious disease agent or other diseases, wherein said vector expresses said antigen. A fragment of the U_{L}29 gene can also be deleted whereby inserting a heterologous sequence enables a fusion protein to be expressed comprising a fragment of the U_{L}29 gene product and heterologous gene product.

In certain embodiments of the invention, any portion of the U_{L}29 gene is deleted and a heterologous sequence is inserted replacing said deleted portion, whereby a fusion protein is expressed comprising a fragment of the U_{L}29 gene product and heterologous gene product.

In another embodiment, the invention provides for a replication defective recombinant mutant herpes simplex virus replication defective vector which can infect mammalian cells and is comprised of at least about one heterologous gene inserted into a region whereby the entire U_{L}29 gene, or fragments thereof, are deleted and are non essential for viral replication, wherein, either:

(i) the heterologous gene is linked to either a fragment of said U_{L}29 gene; or,

(ii) the heterologous gene is operably linked to a promoter which controls its expression; or,

(iii) the heterologous gene comprises sequences from the U_{L}29 gene which are controlled by the HSV promoters, whereby, a fusion protein comprising the heterologous sequence gene product and at least a portion of the U_{L}29 gene product is expressed.

The HSV replication defective vector of the present invention comprises a heterologous sequence which encodes for, and expresses a specific antigen for, a disease and elicits an immune response when administered to a patient in need of treatment. The patient can be immunologically naïve with respect to the antigen expressed by the HSV replication defective vector or can have been pre-immunized. The immune response generated can be humoral and/or cellular and can be directed to infectious disease causing agent such as virus, bacteria, parasites, protozoa and fungi, or any other disease.

Other aspects of the invention are described infra.

Definitions

Prior to setting forth the invention, it may be helpful to an understanding thereof to set forth definitions of certain terms that will be used hereinafter.

A “vector” is a composition which can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

The terms “nucleic acid molecule” or “polynucleotide” will be used interchangeably throughout the specification, unless otherwise specified. As used herein, “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

As used herein, the terms “gene product” or “expression product” refers to the polypeptide encoded by the gene of interest. Expression of the polypeptide can be detected by a number of methods to one of ordinary skill in the art, such as RIA, ELISA, FACS, T-cell proliferation assays, cytotoxic T cell assays and the like.

The term “variant,” when used in the context of a polynucleotide sequence, may encompass a polynucleotide sequence related to a wild type gene. This definition may also include, for example, “allelic”, “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target genes. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer. As used herein, the term “heterologous” is used to refer to a nucleotide sequence which is not normally or naturally found in the specified position within the U_{L}29 gene region. It may therefore be any sequence of nucleotides different from the sequence of the fragment found naturally in the U_{L}29 gene region of the herpes simplex virus and preferably codes for antigenic epitopes which can elicit an immune response against a pathogen or any infectious disease causing agent. As used in relation to a herpes virus “heterologous” may be used to refer to a non-herpes viral sequence, or a sequence not of the specific herpes virus in question. Possible alternative terminology includes “foreign” or “exogenous”. A heterologous nucleotide sequence may encode a sequence of amino acids, i.e. a peptide or a polypeptide.

As used herein, the singular forms “a”, “an” and “the” include plural referents unless the context clearly dictates otherwise.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

“Gene delivery,” “gene transfer,” and the like as used herein, are terms referring to the introduction of an exogenous polynucleotide (sometimes referred to as a “transgenes”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

A significant “fragment” in a nucleic acid context is a contiguous segment of at least about 17 nucleotides, generally at least 20 nucleotides, more generally at least 23 nucleotides, ordinarily at least 26 nucleotides, more ordinarily at least 29 nucleotides, often at least 32 nucleotides, more often at least 35 nucleotides, typically at least 38 nucleotides, more typically at least 41 nucleotides, usually at least 44 nucleotides, more usually at least 47 nucleotides, preferably at least 50 nucleotides, more preferably at least 53 nucleotides, and in particularly preferred embodiments will be at least 56 or more nucleotides.

Homologous nucleic acid sequences, when compared, exhibit significant sequence identity or similarity. The standards for homology in nucleic acids are either measures for homology generally used in the art by sequence comparison or based upon hybridization conditions. The hybridization conditions are described in greater detail below.

As used herein, “substantial homology” in the nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more, typically at least 77%, usually at least 80%, more usually at least about 85%, preferably at least about 90%, more preferably at least about 95 to 98% or more, and in particular embodiments, as high at about 99% or more of the nucleotides. Typically, selective hybridization will occur when there is at least about 55% homology over a stretch of at least about 14 nucleotides, preferably at least about 65%, more preferably at least about 75%, and most preferably at least about 90%. See Kanehisa (1984) Nuc. Acids Res. 12:203-213. The length of homology comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides. The endpoints of the segments may be at many different pair combinations.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

The terms “treatment”, “treating”, and the like are used herein to generally mean obtaining a desired pharmacological and/or physiological effect. The effect may be prophylactic in terms of completely or partially preventing a disease or symptom thereof and/or may be therapeutic in terms of a partial or complete cure of a disease and/or adverse effect attributed to the disease. “Treatment” as used herein covers any treatment of such a disease in a mammal, particularly a human, and includes:

(a) Preventing and/or diagnosing the disease in a subject which may be predisposed to the disease but has not yet been diagnosed as having it;

(b) Inhibiting the disease, i.e. arresting it's development; and/or

(c) Relieving the disease, i.e. causing regression of the disease.

More specifically, “treatment” is intended to mean providing a therapeutically detectable and beneficial effect on a patient suffering from and infectious disease organism, cancer and the like. That effect can include stimulating the patient's own immune system to aid in treating the a patient suffering from or susceptible to, for example, an infectious disease organism, such as HIV, HPV, HBV etc. As used herein, “ameliorated” or “treatment” refers to a symptom which is approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests. For example, amelioration or treatment of a patient suffering from an infectious disease organism, such as for example, Hepatitis B Virus. Amelioration of the disease is measured by a decrease of viral particles in a sample taken from a patient, as measured by, for example, plaque forming units (p.f.u.).

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

As used herein, the term “infectious agent” refers to an organism wherein growth/multiplication leads to pathogenic events in humans or animals. Examples of such agents are: bacteria, fungi, protozoa and viruses.

As used herein, a “pharmaceutically acceptable” component is one that is suitable for use with humans and/or animals without undue adverse side effects (such as toxicity, irritation, and allergic response) commensurate with a reasonable benefit/risk ratio.

“Immune effector cells” refers to cells capable of binding an antigen and which mediate an immune response. These cells include, but are not limited to, T cells (T lymphocytes), B cells (B lymphocytes), monocytes, macrophages, natural killer (NK) cells and cytotoxic T lymphocytes (CTLs), for example CTL lines, CTL clones, and CTLs from tumor, inflammatory, or other infiltrates.

“Activity”, “activation”, “stimulation” or “augmentation” is the ability of immune cells to respond and exhibit, on a measurable level, an immune function. Measuring the degree of activation or “strong immune response” refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

Immune activity or strength of an immune response that may be measured include, but is not limited to, (1) cell proliferation by measuring the cell or DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

As used herein, “specific immune responses” are those immune responses which are specific for a specific antigen. Specificity for an antigen is measured, for example, by the above mentioned assays. The specific immune response is comprised of, for example, B and T lymphocytes and include both cellular and humoral responses (immunoglobulin).

As used herein, “durable immune response” is a specific immune response to an antigen, which is encoded by the vectors described herein, whereby the immune response is measurable in a subject or patient for at least about 1 year, preferably about 2 years, more preferably about 5 years most preferably about 10 years. The immune response can be cellular, T- and/or B-lymphocytes and/or humoral, as measured by in vitro assays such as T cell proliferation assays, cytotoxic assays, ELISA, RIA, gels, FACS analysis, Western Blot and the like.

An immune response is “diminished” if immune activity decreases by about 60%, more preferably at least about 50%, most preferably about 40%, 30%, 20% or 10% as compared to the highest immune activity measured during the treatment of a patient with the herpes replication defective virus vectors of the invention. The percentage decrease is measured as a difference from a normalized value as determined using routine statistical tests.

An “antigen” is any substance that reacts specifically with antibodies or T lymphocytes (T cells). An “antigen-binding site” is the part of an immunoglobulin molecule that specifically binds an antigen. Additionally, an antigen-binding site includes any such site on any antigen-binding molecule, including, but not limited to, an MHC molecule or T cell receptor. “Antigen processing” refers to the degradation of an antigen into fragments (e.g., the degradation of a protein into peptides) and the association of one or more of these fragments (e.g., via binding) with MHC molecules for presentation by “antigen-presenting cells” to specific T cells.

A “co-stimulatory molecule” encompasses any single molecule or combination of molecules which, when acting together with a peptide/MHC complex bound by a T cell receptor on the surface of a T cell, provides a co-stimulatory effect which achieves activation of the T cell that binds the peptide.

“Cells of the immune system” or “immune cells” as used herein, is meant to include any cells of the immune system that may be assayed, including, but not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“T cells” or “T lymphocytes” are a subset of lymphocytes originating in the thymus and having heterodimeric receptors associated with proteins of the CD3 complex (e.g., a rearranged T cell receptor, the heterodimeric protein on the T cell surfaces responsible for antigen/MHC specificity of the cells). T cell responses may be detected by assays for their effects on other cells (e.g., target cell killing, macrophage, activation, B-cell activation) or for the cytokines they produce.

“CD4” is a cell surface protein important for recognition by the T cell receptor of antigenic peptides bound to MHC class II molecules on the surface of an APC. Upon activation, naïve CD4 T cells differentiate into one of at least two cell types, Th1 cells and TH2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response.

“CD8” is a cell surface protein important for recognition by the T cell receptor of antigenic peptides bound to MHC class I molecules. CD8 T cells usually become “cytotoxic T cells” or “killer T cells” and activate macrophages. Effector molecules include, but are not limited to, perforin, granzymes, Fas ligand, IFN-γ, TNF-α, and TNF-β.

“Dendritic cells” (DC) are potent antigen-presenting cells, capable of triggering a robust adaptive immune response in vivo. It has been shown that activated, mature DC provide the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC” defined above) class I or II protein on the surface of APCs. The second type of signal, called a co-stimulatory signal, is neither antigen-specific nor MHC-restricted, and can lead to a full proliferation response of T cells and induction of T cell effector functions in the presence of the first type of signals. This two-fold signaling can, therefore, result in a vigorous immune response. As noted supra, in most non-avian vertebrates, DC arise from bone marrow-derived precursors. Immature DC are found in the peripheral blood and cord blood and in the thymus. Additional immature populations may be present elsewhere. DC of various stages of maturity are also found in the spleen, lymph nodes, tonsils, and human intestine. Avian DC may also be found in the bursa of Fabricius, a primary immune organ unique to avians. In a preferred embodiment, the dendritic cells of the present invention are mammalian, preferably human, mouse, or rat.

An “adjuvant” is any substance capable of enhancing the immune response to an antigen with which it is mixed. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol, as well as BCG (bacilli Calmette-Guerin) and Corynabacterium parvum, which are often used in humans, and ligands of CCR6 and other chemokine receptors.

A “chemokine” is a small cytokine involved in the migration and activation of cells, including phagocytes and lymphocytes, and plays a role in inflammatory responses.

A “cytokine” is a protein made by a cell that affect the behavior of other cells through a “cytokine receptor” on the surface of the cells the cytokine effects. Cytokines manufactured by lymphocytes are sometimes termed “lymphokines.”

By “immunologically effective” is meant an amount of the peptide or fragment thereof which is effective to activate an immune response to prevent or treat proliferative cell growth disorders, such as cancer. Obviously, such amounts will vary between species and individuals depending on many factors. For example, higher doses will generally be required for an effective immune response in a human compared with a mouse.

As used herein, the term “polypeptide” also encompasses amino acid chains of any length, including full length proteins containing the sequences recited herein. A polypeptide comprising an epitope of a protein containing a sequence as described herein may consist entirely of the epitope, or may contain additional sequences. The additional sequences may be derived from the native protein or may be heterologous, and such sequences may (but need not) possess immunogenic or antigenic properties.

An “epitope”, as used herein, is a portion of a polypeptide that is recognized (i.e., specifically bound) by a B-cell and/or T-cell surface antigen receptor. Epitopes may generally be identified using well known techniques, such as those summarized in Paul, Fundamental Immunology, 3rd ed., 243-247 (Raven Press, 1993) and references cited therein. Such techniques include screening polypeptides derived from the native polypeptide for the ability to react with antigen-specific antisera and/or T-cell lines or clones. An epitope of a polypeptide is a portion that reacts with such antisera and/or T-cells at a level that is similar to the reactivity of the full length polypeptide (e.g., in an ELISA and/or T-cell reactivity assay). Such screens may generally be performed using methods well known to those of ordinary skill in the art, such as those described in Harlow and Lane, Antibodies: A Laboratory Manual, Cold Spring Harbor Laboratory, 1988. B-cell and T-cell epitopes may also be predicted via computer analysis. Polypeptides comprising an epitope of a polypeptide that is preferentially expressed in a tumor tissue (with or without additional amino acid sequence) are within the scope of the present invention.

The terms “specific binding” or “specifically binding”, as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope “A”, the presence of a protein comprising epitope A (or free, unlabeled A) in a reaction comprising labeled “A” and the antibody will reduce the amount of labeled A bound to the antibody. “Specific binding” in general, refers to any immune related molecule binding to its ligand, such as for example the binding of a T cell receptor expressed by a T lymphocyte, to an MHC molecule and peptide on an antigen presenting cell.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

### BRIEF DESCRIPTION OF THE DRAWINGS

FIG. 1 is a schematic representation of the HSV-1 recombinant mutants.

FIGS. 2A and 2B are graphs showing the results of antibody responses as a function of viral dose in mice immunized with various doses of the replication defective mutants.

FIG. 2A shows the antibody titers of HSV.

FIG. 2B shows the antibody titers of β-galactosidase.

FIGS. 3A to 3D are graphs showing the induction and durability of IgG in mice immunized with the replication defective mutants.

FIGS. 3A and 3B are graphs showing the induction of IgG specific for HSV-1 and β-galactosidase respectively.

FIGS. 3C and 3D are graphs showing the durability of the IgG responses to HSV-1 and β-galactosidase.

FIGS. 4A to 4B are graphs showing the induction of β-galactosidase IgG after two HSV-1 immunizations.

FIG. 4A is a graph showing the titers of IgG specific for HSV-1.

FIG. 4B is a graph showing the titers of IgG specific for β-galactosidase.

FIGS. 5A to 5D are graphs showing the induction of β-galactosidase IgG by cell-free HD-2 virus after one or two immunizations.

FIG. 5A is a graph showing the titers of IgG specific for HSV-1.

FIG. 5B is a graph showing the titers of IgG specific for β-galactosidase.

FIG. 5C is a graph showing the titers of IgG specific for HSV-1 after being immunized with twice with d301 followed by cell-free HD-2 virus immunizations.

FIG. 5D is a graph showing the titers of IgG specific for β-galactosidase after being immunized twice with d301 followed by cell-free HD-2 virus immunizations.

FIGS. 6A to 6B are graphs showing the induction of β-galactosidase IgG by cell-free HD-2 virus after immunization with replication competent HSV-1.

FIG. 6A is a graph showing the titers of IgG specific for HSV-1.

FIG. 6B is a graph showing the titers of IgG specific for β-galactosidase.

FIG. 7 is a graph showing cellular proliferative responses to β-galactosidase in immune mice.

### DETAILED DESCRIPTION OF THE INVENTION

The efficacy of replication-defective HSV-derived replication defective vectors was evaluated by measuring the generation and durability of immune responses, both humoral and cellular. The efficacy of HSV vectors described herein, provide a durable immune response even if there is prior HSV immunity, as determined by the ability of immune mice to generate β-galactosidase-specific IgG responses. These results differ from the suppression that has been described for previously described vectors. In addition, these HSV vectors elicit a durable immunity, characterized by HSV-specific and β-galactosidase-specific IgG antibody responses that are maintained for at least 1 year following vector administration. These properties of HSV as a vector are very useful in the generation of vaccines for immunizing mammals against pathogens and other diseases.

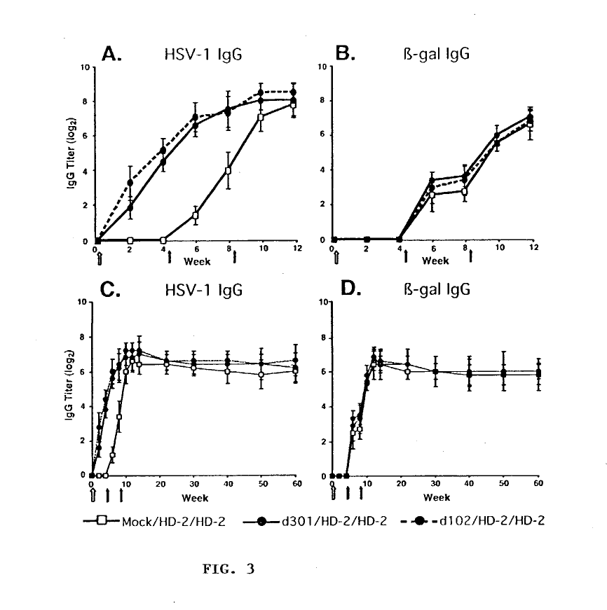

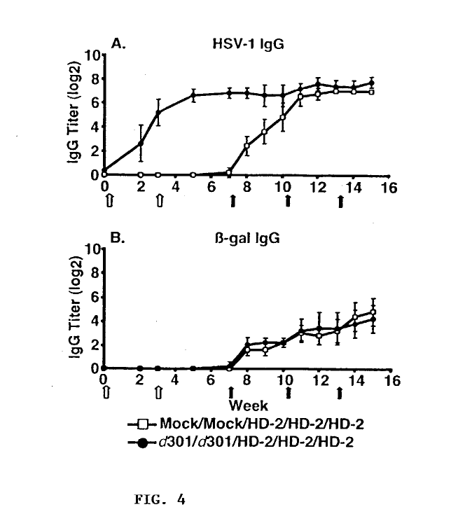

The vectors described herein are advantageous in many respects and differ to known vectors. With respect to efficacy, the recombinant, replication defective herpes simplex virus (HSV) vector of this invention is highly efficacious at inducing T cells and antibodies to the inserted heterologous protein expressed by the virus. For example, a recombinant, replication defective HSV vector comprising a sequence encoding the β-galactosidase gene as the heterologous gene, was administered to animals by a subcutaneous route (see Examples which follow), had an IgG titer to β-galactosidase up to 60 weeks after immunization as shown if FIGS. 3A and 3B. Use of the β-galactosidase gene is merely illustrative of the heterologous gene and is not meant to limit or construe the invention to one type of gene. Any heterologous gene product that can elicit an immune response is a candidate for use in the replication defective HSV of the present invention and, thus, is a potential candidate for treatment of a disease.

The replication defective HSV vectors of this invention are surprisingly more effective as a vaccine than other, previously reported, vaccines in the generation of, and duration of, an immune response in both naïve and pre-immunized individuals. In contrast to the other replication defective vectors, such as for example adenovirus based vectors, the HSV replication defective vector composition useful in the present invention can be used at lower doses due to its ability to maintain a persistent infection. Preferred embodiments of this HSV replication defective vector can also be administered in a single inoculation to obtain substantially complete protection.

The replication defective mutant HSV replication defective vector is also highly advantageous in that a pre-existing HSV immunity is not detrimental to the efficacy of preferred of the present invention, as described in detail in the Examples which follow. The replication defective HSV is also highly useful as it can be used as a prophylactic or therapeutic vaccine against any pathogen for which the antigen(s) crucial for induction of an immune response able to limit the spread of the pathogen has been identified and for which the cDNA is available.

The heterologous nucleic acid encodes a protein which is desirably capable of inducing an immune response to a pathogen or infectious disease causing agent. Such a protein may be a protein from another herpes virus, especially genital herpes virus, rabies virus, human papilloma virus, human immunodeficiency virus (HIV), respiratory syncytial virus (RSV). As an example, the replication defective HSV mutant comprises at least about one heterologous gene sequence which codes for antigens useful in treating diseases such as for example, HIV gp120 protein, influenza virus nucleoproteins and envelope proteins, or any other antigenic epitopes that elicit an immune response, humoral and/or cellular, and result in the amelioration of the disease. A vector comprising at least about 2, 3, 4, or 5 heterologous sequences is within the scope of the present invention. The heterologous sequences can be from different antigenic epitopes of an antigen or can be derived from more than one species or genus. The HSV replication defective vector method of the present invention also can be employed with a tumor-associated protein specific for a selected malignancy. These tumor antigens include viral oncogenes, such as E6 and E7 of human papilloma virus or cellular oncogenes such as mutated ras or p53. Particularly, where the condition is human immunodeficiency virus (HIV) infection, the protein is preferably HIV glycoprotein 120 for which sequences are available from GenBank. Where the condition is human papilloma virus infection, the protein is selected preferably from the group consisting of E6, E7 and/or L1 [Seedorf, K. et al, Virol., 145:181-185 (1985)]. Where the condition is respiratory syncytial virus infection, the protein is selected preferably from the group consisting of the glyco-(G) protein and the fusion (F) protein, for which sequences are available from GenBank. In addition to these proteins, other virus-associated proteins are readily available to those of skill in the art. Selection of the particular heterologous proteins is not a factor in this invention.

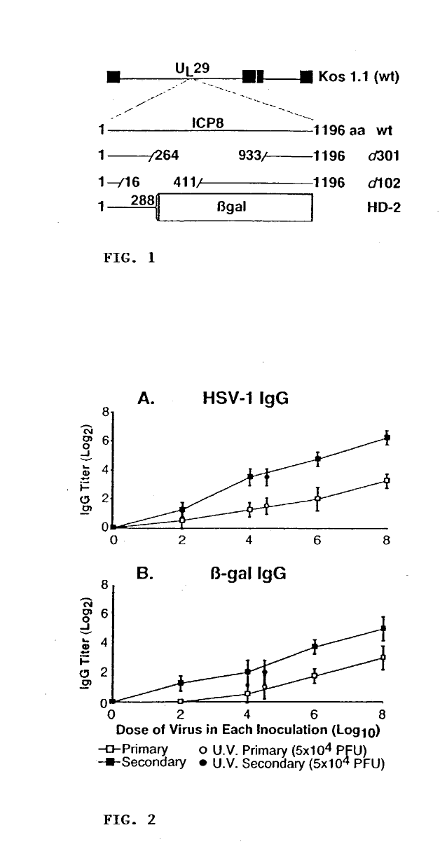

The HSV replication defective vectors of the invention were prepared as shown in FIG. 1 and as described in Gao, M., and D. M. Knipe. 1989. “Genetic evidence for multiple nuclear functions of the herpes simplex virus ICP8 DNA-binding protein”. Journal of Virology. 63:5258-5267, which is incorporated herein, in its entirety. Briefly, the following example is provided. This example is not meant to be limiting or construed in any way but serves merely to illustrate the construction of a replication defective HSV vector. Plasmids such as pUC were used as cloning vectors. Plasmid, pd301 was generated by an internal in-frame deletion of a 2,001 base pair NotI fragment (nucleic acid positions 1395 to 3396) of the UL₂₉ gene. The plasmid was cotransfected with wild type HSV 1 DNA (KOS 1.1 strain; described by Knipe, D. M. et al; 1982. J. Virol. 43:314-324; which is incorporated in its entirety, herein) into a neomycin resistant cell line. Progeny viruses are selected for their ability to grow in neomycin resistant cell lines.

As an alternative illustrative example, d102 deletion mutant shown in FIG. 1, is constructed by partially digesting a plasmid comprising a SmaI site and ligating into the plasmid a BglII linker of about 12 base pairs. A 1,188 base pair deletion of the UL₂₉ gene is generated by digestion with BglII restriction enzyme to yield plasmid pd102 lacking nucleic acid bases at positions 17 through to 411 of the UL₂₉ gene. A recipient virus is prepared using a plasmid comprising a deletion of a 780 base pair XhoI fragment, a BglII linker and a lacZ gene (Pharmacia Inc., Piscataway, N.J.). The plasmid is co-transfected with KOS1.1 DNA into a neomycin resistant cell line. Recombinant viruses were identified as blue plaques after incubation with X-Gal. One mutant virus was plaque purified and designated as HD-2. Thus, replication defective vectors can be obtained using restriction enzymes to excise specific regions of a gene required for replication, insertion of a polynucleotide that encodes for an antigen of choice and co-transfect cells with a wild type strain of virus and select for deletion mutant virus vectors as described by Gao, M., and D. M. Knipe. 1989. Journal of Virology. 63:5258-5267.

Methods for the construction of engineered viruses are known in the art. Additional methods for the genetic manipulation of DNA sequences are known in the art. Generally, these include Ausubel et al., chapter 16 in CURRENT PROTOCOLS IN MOLECULAR BIOLOGY (John Wiley and Sons, Inc.): U.S. Pat. No. 4,603,112, Paoletti et al., issued July 1986. Virological considerations also are reviewed in Coen D. M., “Molecular Genetics of Animal Viruses,” in VIROLOGY 123-150 (2nd ed.) (Raven Press, 1990). The construction of HSV-1 vectors is described, for example, in U.S. Pat. No. 5,288,641; Roizman and Jenkins, J. Science 229: 1208 (1985); Johnson et al., J. Virol. 66: 2952 (1992); Gage et al., J. Virol. 66: 5509 (1992); Spaete and Frenkel, Cell 30; 295 (1982); Goldstein and Weller, J. Virol. 62: 196 (1988), Coen, chapter 7, Virology, Raven Press, 1990; Breakefield and DeLuca, The New Biologist, 3: 203 (1991); Leib and Olivo, BioEssays 15: 547 (1993); Glorioso et al., Seminars in Virology 3: 265 (1992); Chou and Roizman. Proc. Natl. Acad Sci. USA, 89: 3266 (1992); Breakfield et al., Molec. Neurobiol. 1: 339 (1987); Shih et al., in: VACCINES 85, Cold Spring Harbor Press (1985) 177-180; Palella et al., Molec. Cell. Biol. 8: 457 (1988): Matz et al., J. Gen. Virol. 64: 2261 (1983); Mocarski et al., Cell 22: 243 (1980); Coen et al., Science 234: 53 (1986) and Lee, C. K., and Knipe D. M. (1983) J. Virol. 46:909-919.

The ICP8 or any gene that renders the HSV vector replication defective such as immediate early genes, or early genes may be rendered functionally inactive by several techniques well known in the art. For example, they may be rendered functionally inactive by deletions, substitutions or insertions, preferably by deletion. Deletions may remove portions of the genes or the entire gene. Inserted sequences may include the heterologous genes described infra.

Mutations are made in the HSV strains by homologous recombination methods well known to those skilled in the art. For example, HSV genomic DNA is transfected together with a vector, preferably a plasmid vector, comprising the mutated sequence flanked by homologous HSV sequences. The mutated sequence may comprise deletions, insertions or substitutions, all of which may be constructed by routine techniques. Insertions may include selectable marker genes, for example lacZ, for screening recombinant viruses by, for example, β-galactosidase activity.

In a preferred embodiment, the mutant HSV replication defective vectors of the invention can be modified to carry a heterologous gene, that is to say a gene other than one present in the HSV genome. The term “gene” is intended to cover at least sequences which are capable of being transcribed, optionally with some or all of 5′ and/or 3′ transcribed but untranslated flanking sequences naturally associated with the translated coding sequence. It may optionally further include the associated transcriptional and/or translational control sequences normally associated with the transcribed sequences. The heterologous gene is preferably inserted into the region of the ICP8 gene. The heterologous gene may be inserted into the HSV genome by homologous recombination of HSV strains with, for example, plasmid vectors carrying the heterologous gene flanked by HSV sequences. The heterologous gene may be introduced into a suitable plasmid vector comprising HSV sequences using cloning techniques well-known in the art.

The transcribed sequence of the heterologous gene is preferably operably linked to a control sequence permitting expression of the heterologous gene in mammalian cells, preferably cells of the central and nervous system. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence.

The control sequence comprises a promoter allowing expression of the heterologous gene and a signal for termination of transcription. The promoter is selected from promoters which are functional in mammalian, preferably human, cells. The promoter may be derived from promoter sequences of eukaryotic genes. For example, it may be a promoter derived from the genome of a cell in which expression of the heterologous gene is to occur, preferably a cell of the mammalian central or peripheral nervous system. With respect to eukaryotic promoters, they may be promoters that function in a ubiquitous manner (such as promoters of a-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter. The HSV LAT promoter, and promoters comprising elements of the LAT promoter region, can also be used.

In addition, any of these promoters may be modified by the addition of further regulatory sequences, for example enhancer sequences. Chimeric promoters may also be used comprising sequence elements from two or more different promoters described above, for example an MMLV LTR/LAT fusion promoter or promoters comprising elements of the LAT region.

The heterologous gene may encode, for example, antigens from infectious disease organisms, tumors, proteins involved in the regulation of cell division, for example mitogenic growth factors including neurotrophic growth factors (such as brain-derived neurotrophic factor, glial cell derived neurotrophic factor, NGF, NT3, NT4 and NT5, GAP43 and), cytokines (such as α-,β- or γ-interferon, interleukins including IL-1, IL-2, tumor necrosis factor, or insulin-like growth factors I or II), protein kinases (such as MAP kinase), protein phosphatases and cellular receptors for any of the above. The heterologous gene may also encode enzymes involved in cellular metabolic pathways, for example enzymes involved in amino acid biosynthesis or degradation (such as tyrosine hydroxylase), purine or pyrimidine biosynthesis or degradation, and the biosynthesis or degradation of neurotransmitters, such as dopamine, or protein involved in the regulation of such pathways, for example protein kinases and phosphatases. The heterologous gene may also encode transcription factors or proteins involved in their regulation, for example members of the Brn3 family (including Brn-3a, Brn-3b and Brn-3c) or pocket proteins of the Rb family such as Rb or p107, membrane proteins (such as rhodopsin), structural proteins (such as dystrophin) or heat shock proteins such as hsp27, hsp65, hsp70 and hsp90.

Preferably, the heterologous gene encodes a polypeptide of therapeutic use. For example, of the proteins described above, tyrosine hydroxylase can be used in the treatment of Parkinson's disease, rhodopsin can be used in the treatment of eye disorders, dystrophin may be used to treat muscular dystrophy, and heat shock proteins can be used to treat disorders of the heart. Polypeptides of therapeutic use may also include cytotoxic polypeptides such as ricin, or enzymes capable of converting a precursor prodrug into a cytotoxic compound for use in, for example, methods of virus-directed enzyme prodrug therapy or gene-directed enzyme prodrug therapy. In the latter case, it may be desirable to ensure that the enzyme has a suitable signal sequence for directing it to the cell surface, preferably a signal sequence that allows the enzyme to be exposed on the exterior of the cell surface whilst remaining anchored to cell membrane. Suitable enzymes include bacterial nitroreductase such as E. coli nitroreductase as disclosed in WO93/08288 or carboxypeptidase, especially carboxypeptidase CPG2 as disclosed in WO88/07378. Other enzymes may be found by reference to EP-A-415731. Suitable prodrugs include nitrogen mustard prodrugs and other compounds such as those described in WO88/07378, WO89/10140, WO90/02729 and WO93/08288 which are incorporated herein by reference.

In another preferred embodiment, the replication defective mutant herpes simplex virus elicits a durable humoral and cellular immune response specific for the antigen encoded by said herpes virus in both naïve and pre-immunized animals. A vector encoding an antigen of choice for treatment of a disease is administered to a mammal in a therapeutically effective or sufficient amount to treat a mammal suffering from or susceptible to, for example, an infectious disease organism such as Human immunodeficiency virus, Herpes Simplex Virus, Hepatitis-A, -B, -C Virus and the like. Preferably, the vector is a recombinant replication defective mutant Herpes Simplex Virus comprising a herpes simplex virus wherein the entire U_{L}29 gene, or fragments thereof are deleted and, in the site of the deleted U_{L}29 gene or deleted fragments thereof, a heterologous sequence encoding an antigen from a specific infectious disease causing agent is inserted which, when administered to the mammal in the replication defective mutant herpes simplex virus, elicits an immune response against the infectious disease causing agent.

Preferably, the vector stimulates an immune response to ameliorate or eradicate the infectious disease organism or diseases such as cancer, neuronal diseases, and the like. For example, the recombinant vectors provide specific anti-tumor effect for patients who have been diagnosed with, for example, Her2/neu⁺ tumors such as breast, renal, prostate, and other HER2 tumors. However, this antigen is merely an illustrative example and is not meant to be construed as limiting the present invention in any way. Examples of other antigens that are useful for treating different types of cancers include, but are not limited to, overexpressed or mutated forms of antigens. Additional examples include, for instance, carcinogenic embryonic antigen (CEA) for gastrointestinal cancers; K-ras for lung, gastrointestinal and bladder cancers; p53 which affects a wide variety of neoplastic growth; SARDT3 in neck and head cancers.

In another preferred embodiment, the immune response that is stimulated by the vector composition is durable. Preferably, cellular and/or antibodies are measurable in an individual treated with the desired vector expressing an antigen of choice, for at least about 1 year, preferably at least about 2 years, more preferably at least about 5 years, most preferably at least about 10 years. Durability of an immune response is measured by means well known to one skilled in the art and include but not limited to, T cell assays such as proliferation assay, cytotoxic T cell assays, RIA, ELISA, gels, Western Blot, FACS etc.

Without wishing to be bound by theory, potential mechanisms for immune durability such as the durability of the antibody responses induced by the HSV vector (See Examples which follow) could be that the ability of the vector to infect primary cells and the potential of virus-infected cells to persist in vivo may be important determinants for vaccine efficacy. Cellular cytopathic effects induced by the virus may significantly affect the ability of infected cells to maintain long-term antigen expression. The durability of the antibody response generated by HSV may also indicate that this virus is able to readily activate long-lived plasma cells. Alternatively, viral antigens may persist for extended periods on antigen presenting cells or the virus itself may persist at low levels and continue to express small amounts of antigen.

In another preferred embodiment, the vectors induce long term memory immune memory cells (“memory” cells (i.e. previously exposed to a specific antigen). In accordance with the invention, the vector encoding an antigen is administered to a subject. The replication-defective mutant viral vectors preferably infect only one round of cells and do not spread. The antigen encoded by the vector is presented on the surface of the infected cell. The antigen presenting cells process and present antigen to the immune system. The immune system will recognize the peptide fragment that is presented in the context of HLA molecules by engagement with the receptor of an immune effector cell such as, for example a T cell.

Several different ways, to assess maturity, memory cells and cell differentiation, are available. For example, one such method is by measuring cell phenotypes. Immune cells express a variety of cell surface molecules, for example, memory T cells are CD45RO⁺ and can be differentiated from naïve T cells (not previously exposed to a specific antigen) which are CD45RA⁺. The phenotypes of immune cells and any phenotypic changes can be evaluated by flow cytometry after immunofluorescent staining using monoclonal antibodies that will bind membrane proteins characteristic of various immune cell types. Immune cells that have undergone differentiation or activation can also be enumerated by staining for the presence of characteristic cell surface proteins by direct immunofluorescence in fixed smears of cultured cells. The term “fluorescent component” or “fluorescent label” or “labeled” refers to a component capable of absorbing light and then re-emitting at least some fraction of that energy as light over time. The term includes discrete compounds, molecules, naturally fluorescent proteins and macro-molecular complexes or mixtures of fluorescent and non-fluorescent compounds or molecules. The term “fluorescent component” or “fluorescent label” also includes components that exhibit long lived fluorescence decay such as lanthanide ions and lanthanide complexes with organic ligand sensitizes, that absorb light and then re-emit the energy over milliseconds. Other labels include different fluorochromes and fluorescent proteins such as green fluorescent protein. Fluorochromes which may find use in a multicolor analysis include phycobiliproteins, e.g., phycoerythrin and allophycocyanins; fluorescein and Texas red.

As will be recognized by those in the art, the term “host compatible” or “autologous” cells means cells that are of the same or similar haplotype as that of the subject or “host” to which the cells are administered.

The presentation of Class I MHC molecules bound to peptide alone has generally been ineffective in activating CD8 cells. In nature, the CD8 cells are activated by antigen-presenting cells, such as, for example, dendritic cells, which present not only a peptide-bound Class I MHC molecule, but also a costimulatory molecule. Such costimulatory molecules include B7 which is now recognized to be two subgroups designated as B7.1 and B7.2. It has also been found that cell adhesion molecules such as integrins assist in this process.

Dendritic cells are antigen-presenting cells that are found in all tissues and organs, including the blood. Specifically, dendritic cells present antigens for T lymphocytes, i.e., they process and present antigens, and stimulate responses from naive and memory T cells. In addition to their role in antigen presentation, dendritic cells directly communicate with non-lymph tissue and survey non-lymph for an injury signal (e.g., ischemia, infection, or inflammation) or tumor growth. Once signaled, dendritic cells initiate the immune response by releasing IL-1 which triggers lymphocytes and monocytes. When the CD8 T-cell interacts with an antigen-presenting cell, such as a dendritic cell, having the peptide bound by a Class I MHC and costimulatory molecule, the CD8 T-cell is activated to proliferate and becomes an effector T-cell. See, generally, Janeway and Travers, Immunobiology, published by Current Biology Limited, London (1994), incorporated by reference.

Accordingly, what is needed and what the present invention provides, is a means to activate T-cells so that they proliferate, become cytotoxic for cells expressing the desired antigen, such as for example, HIV gp120, and maintain memory cells specific for the administered antigen. Thus, the immune system is primed against various epitopes so that a pool of primed immune cells exists which become activated to recognize and kill cells infected with the heterologous antigen.

A review of the biology of memory T cells may be found in Dutton et al. (1998) Ann. Rev Immunol 16:201-23. Memory cells express a different pattern of cell surface markers, and they respond in several ways that are functionally different from those of naive cells. Human memory cells are CD45RA⁻, CD45RO⁺. In contrast to naïve cells, memory cells secrete a full range of T cell cytokines.

Chemokines and cytokines also play a powerful role in the development of an immune response. The role of chemokines in leukocyte trafficking is reviewed by Baggiolini (1998) Nature 392:565-8, in which it is suggested that migration responses in the complicated trafficking of lymphocytes of different types and degrees of activation will be mediated by chemokines. The use of small molecules to block chemokines is reviewed by Baggiolini and Moser (1997) J. Exp. Med. 186:1189-1191.

The role of various specific chemokines in lymphocyte homing has been previously described. For example, Campbell et al. (1998) Science, showed that SDF-1 (also called PBSF), 6-C-kine (also called Exodus-2), and MIP-3beta (also called ELC or Exodus-3) induced adhesion of most circulating lymphocytes, including most CD4+ T cells; and MIP-3alpha (also called LARC or Exodus-1) triggered adhesion of memory, but not naïve, CD4+ T cells. Tangemann et al. (1998) J. Immunol. 161:6330-7 disclose the role of secondary lymphoid-tissue chemokine (SLC), a high endothelial venule (HEV)-associated chemokine, with the homing of lymphocytes to secondary lymphoid organs. Campbell et al. (1998) J. Cell Biol 141(4):1053-9 describe the receptor for SLC as CCR7, and that its ligand, SLC, can trigger rapid integrin-dependent arrest of lymphocytes rolling under physiological shear.

Induction of mature B cells by the vectors described herein, can be measured in immunoassays, for example, by cell surface antigens including CD19 and CD20 with monoclonal antibodies labeled with fluorochromes or enzymes may be used to these antigens. B cells that have differentiated into plasma cells can be enumerated by staining for intracellular immunoglobulins by direct immunofluorescence in fixed smears of cultured cells.

Several different ways are available to assess maturity and cell differentiation. For example, one such method is by measuring cell phenotypes. The phenotypes of immune cells and any phenotypic changes can be evaluated by flow cytometry after immunofluorescent staining using monoclonal antibodies that will bind membrane proteins characteristic of various immune cell types.

A second means of assessing cell differentiation is by measuring cell function. This can be done biochemically, by measuring the expression of enzymes, mRNA's, genes, proteins, or other metabolites within the cell, or secreted from the cell. Bioassays also can be used to measure functional cell differentiation or measure specific antibody production directed to an infectious disease organism, a patient's tumor, tumor cell lines or cells from fresh tumors.

As used herein, “fresh tumors” refer to tumors removed from a host by surgical or other means.

In vitro T cell cytotoxic assays are well known to those skilled in the art. In general, cytotoxicity is measured in a 5 hr ⁵¹Sodium chromate (⁵¹Cr) release assay. In particular, a 20 hr ⁵¹Cr-release assay is preferred. Tumor cells, also referred to herein as “target cells”, are plated in flat-bottomed microtiter plates and incubated at 37° C. overnight. The Target cells are washed and labeled the next day with ⁵¹Cr at 37° C. ⁵¹Cr is taken up by the target cells, either by endocytosis or pinocytosis, and is retained in the cytoplasm. The wells containing tumor cells are washed, and then armed or unarmed ATC, referred to as “effector cells” are plated at different E:T ratios and incubated overnight at 37° C. Cytolysis is a measure of the ⁵¹Cr released from the target cells into the supernatant due to destruction of the target cells by the effector cells. The microtiter plates are centrifuged at 1000 rpm for 10 minutes and an aliquot of about 50 μl to about 100 μl is removed and the level of radioactivity is measured the next day by a gamma counter and the percent specific lysis calculated.

Percent specific lysis is measured by using the formula:

{{(⁵¹Cr released from the target cells)−(spontaneous ⁵¹Cr released from the target cells)}/{(maximum ⁵¹Cr released from the target cells)−(spontaneous ⁵¹Cr released from the target cells)}}×100

The spontaneous ⁵¹Cr released from the target cells is measured with tumor cells to which no effector cells have been added. Maximum ⁵¹Cr released from the target cells is obtained by adding, for example, 1M HCl and represents the total amount of ⁵¹Cr present in the cytoplasm of the target cell.

Other means of assaying for T lymphocyte activity is by the mixed lymphocyte reaction described in the examples which follow. Other cytotoxicity assays such as the labeling of target cells with tritiated thymidine (³H-TdR) may also be used. ³H-TdR is taken up by target cells into the nucleus of the cell. Release of ³H-TdR is a measure of cell death by DNA fragmentation. The assay is conducted as above except the incubation period is at least about 48 hours and 50 μl to about 100 μl of the supernatant is measured by a beta-counter in the presence of at least about 1 ml of scintillation fluid. Calculation of percent specific lysis is performed using the above formula.

In another preferred embodiment, the vectors of the invention are efficacious at lower doses than vectors described in the prior art. As used herein, “efficacious” is determined by the amelioration of a disease state. Without wishing to be bound by theory, the ability of the HSV vectors to be efficacious is due in part to immune evasion properties of HSV. HSV encodes a number of immune modulatory proteins that may aid in the protection of virions or virus-infected cells from immune mediated clearance. For example, the interaction of glycoprotein C (gC) with the complement component C3 has been shown to protect virions from complement-dependent neutralization and to defend virus-infected cells from complement-mediated lysis. Binding of HSV-1 gC to mouse C3 has been observed in vitro, consistent with a role for gC in enhancing primary infection or persistence of virus infected cells in this system. In addition, a heterodimer of glycoproteins E (gE) and I (gI) forms an Fc gamma receptor that has been reported to protect virus infected cells from antibody-dependent cellular cytotoxicity.

In accordance with preferred embodiments of the invention, the inserted heterologous sequence comprises a promoter which directs the expression of DNA encoding an antigen from a specific infectious disease causing agent, or the inserted sequence coding for an antigen from a specific infectious disease causing agent is under the control of the herpes simplex virus promoter.

In a particularly preferred embodiment of the invention, prior host immunity to herpes simplex virus does not diminish the efficacy of the replication defective mutant herpes simplex virus vector and multiple inoculations using the same replication defective herpes simplex virus does not result in a diminished immune response to the antigen encoded by the vector. Preferably, at least about one inoculation is administered, more preferably at least about 2 inoculations are administered, most preferably at least about 3 inoculations are administered, without diminishing the efficacy or durability of the immune response to the desired antigen.

In accordance with additional embodiments of the invention, multiple vectors encoding different antigenic peptides can be administered simultaneously. The gene product of the inserted sequence is not limited to one antigenic epitope but can comprise one or more antigenic epitopes, either from the same antigenic peptide or can be from multiple peptides which may not be from the same genus. For example, the vector can comprise heterologous sequences from more than one infectious disease agent. Alternatively, the vector can encode for variants or mutants of a wild type antigen. The compositions and methods of the present invention encompass variants of wild type polypeptides and nucleic acid sequences encoding such polypeptides. A polypeptide “variant,” as used herein, is a polypeptide that differs from the native polypeptide in substitutions and/or modifications, such that the antigenic and/or immunogenic properties of the polypeptide are retained. Such variants generally can be identified by modifying one of the above polypeptide sequences and evaluating the reactivity of the modified polypeptide with antisera and/or T-cells as described above. Nucleic acid variants can contain one or more substitutions, deletions, insertions and/or modifications such that the antigenic and/or immunogenic properties of the encoded polypeptide are retained. One preferred variant of the polypeptides described herein is a variant that contains nucleotide substitutions, deletions, insertions and/or modifications at no more than 20% of the nucleotide positions.

In general, nucleotide sequences encoding all or a portion of polypeptides can be prepared using any of several techniques well known to those skilled in the art. For example, cDNA molecules encoding such polypeptides can be cloned on the basis of, for example, the breast tumor-specific expression of the corresponding mRNAs, using differential display PCR. This technique compares the amplified products from RNA template prepared from normal and breast tumor tissue. cDNA can be prepared by reverse transcription of RNA using a random primer, such as for example, (dT)₁₂ AG primer. Following amplification of the cDNA using a random primer, a band corresponding to an amplified product specific to the tumor RNA may be cut out from a silver stained gel and subcloned into a suitable vector, such as the HSV vector described in the examples which follow.

In another preferred embodiment, the replication defective mutant herpes simplex virus can be used in a series of inoculations with at least about one other vaccine. The vaccine and the vector can induce the same specific immune responses to the same antigen or each can elicit an immune response to different epitopes of the antigen.

In another preferred embodiment, the replication defective mutant herpes simplex virus is used to increase the durability of immune responses induced by said vaccine(s) by immunizing with said vaccine(s), followed by at least about one booster immunization with said replication defective mutant herpes simplex virus.

In another embodiment, the vector comprises a replication defective herpes virus vector comprising a deleted U_{L}29 gene or fragments thereof, and inserting into said deleted U_{L}29 gene, a heterologous sequence encoding an antigen from a specific infectious disease agent or other diseases, wherein said vector expresses said antigen. A fragment of the U_{L}29 gene can also be deleted and inserting a heterologous sequence whereby, a fusion protein is expressed comprising a fragment of the U_{L}29 gene product and heterologous gene product.

In certain embodiments, any portion of the U_{L}29 gene is deleted and inserting a heterologous sequence in said deleted portion, whereby a fusion protein is expressed comprising a fragment of the U_{L}29 gene product and heterologous gene product.

In another embodiment, the invention provides for a replication defective recombinant mutant herpes simplex virus (HSV) replication defective vector which can infect mammalian cells and is comprised of at least about one heterologous gene inserted into a region whereby the entire U_{L}29 gene, or fragments thereof, are deleted and are non essential for viral replication wherein, either:

(i) the heterologous gene is linked to either a fragment of said U_{L}29 gene; or,

(ii) the heterologous gene is operably linked to a promoter which controls its expression; or,

(iii) the heterologous gene comprises sequences from the U_{L}29 gene which are controlled by the HSV promoters, whereby, a fusion protein comprising the heterologous sequence gene product and U_{L}29 gene product is expressed.

The vector comprises a heterologous sequence which encodes for, and expresses a specific antigen for, a disease and elicits an immune response when administered to a patient in need of treatment. The patient can be immunologically naïve with respect to the antigen expressed by the or has been pre-immunized. The immune response generated can be humoral and/or cellular and can be directed to infectious disease causing agent such as virus, bacteria, parasites, protozoa and fungi, or any other disease.

In a particular preferred embodiment, the replication defective HSV vector is protective against infections from other viruses. Particularly preferred viral organisms causing human diseases according to the present invention include (but not restricted to) Herpes viruses, Hepatitisviruses, Retroviruses, Orthomyxoviruses, Paramyxoviruses, Togaviruses, Picomaviruses, Papovaviruses and Gastroenteritisviruses.

According to another preferred embodiment of the invention, the replication defective HSV vector provides protective functions against infections by human or domestic animal bacterial pathogens. Particularly preferred bacteria causing serious human diseases, and which are useful in the practice of the present invention, are the Gram positive organisms: Staphylococcus aureus, Staphylococcus epidermidis, Enterococcus faecalis and E. faecium, Streptococcus pneumoniae and the Gram negative organisms: Pseudomonas aeruginosa, Burkholdia cepacia, Xanthomonas maltophila, Escherichia coli, Enterobacter spp, Klebsiella pneumoniae and Salmonella spp.

As used herein, “protective functions” refers to the ability of the replication defective HSV vector to generate an immune response against the infecting pathogen or other diseases resulting in the amelioration of the disease state.

As used herein, “normal subject” refers to any mammal of the same species, age, sex, which is not exposed to, or infected by, an infectious agent or suffering from any disease, and has normal cytokine profiles and is within the parameters of a healthy subject as defined by medical and veterinarian guidelines and text books.

Embodiments of the invention include the use of viruses such as for example replication defective viruses to obtain long-term gene expression in latently infected cells. In a most preferred embodiment the viral vector used is a HSV vector or most preferred, an HSV-derived vector, e.g. a mutant which is unable to initiate the cycle of productive infection and which may therefore be driven into the latent state or a mutant able to establish latency in cell-types other than neurons. Replication defective and/or attenuated viruses may be preferred for certain purposes. As used herein, “vector”, “construct” or “replication defective virus” are used interchangeably and refer to the replication defective herpes simplex virus of the invention as described in detail in the Examples section which follow.

A replication defective herpes virus (HSV) can lack a functional form of one or more of the regulatory proteins ICP0, ICP4, ICP8, ICP22, ICP27 and ICP47, and may lacking additionally or alternatively the essential glycoprotein H (gH) gene, or a functional form thereof. Most preferably, the gene encoding the ICP8 is deleted in its entirety or at least fragments thereof. Propagation of a replication defective virus requires a complementing cell line. Cell lines able to complement both ICP8 and ICP27 gene defects are the Vero-derived cell line V8-27, Da Costa et al., Virology (2001) 288:256-263.

Cells comprising a Herpes Simplex Virus replication defective vector according to the present invention are provided as a further aspect of the invention, especially cells in which the Herpes Simplex Virus replication defective vector is incorporated. Such cells may be in culture in vitro, and useful for study of expression etc., or may be cells from a mammal, especially human or a non-human mammal such as a-primate or rodent such as a mouse.

Methods which comprise introduction of a Herpes Simplex Virus replication defective vector according to the present invention into a cell, e.g. by means of viral infection, are also provided. This can be performed ex vivo (in vitro) or in vivo. A method according to the present invention may include causing or allowing expression of a heterologous nucleotide sequence in a nucleic acid Herpes Simplex Virus replication defective vector, for instance, within a cell following introduction of the Herpes Simplex Virus replication defective vector into the cell or an ancestor thereof. A cell containing a Herpes Simplex Virus replication defective vector according to the invention, e.g., as a result of introduction of the Herpes Simplex Virus replication defective vector into the cell or an ancestor thereof, can be administered to a subject. Following such introduction of the Herpes Simplex Virus replication defective vector into the cell, cells can be cultured or maintained ex vivo and then delivered to a subject, either from which they were obtained (or from which an ancestor was obtained) or a different subject. Where cells are to be used as an immunogen (for instance), they may be killed or inactivated prior to administration.

Also provided is a method comprising administration of a composition comprising a Herpes Simplex Virus replication defective vector, as disclosed, to an individual. The administration can be by infection with a viral vector which comprises the Herpes Simplex Virus replication defective vector. Naked DNA delivery can be used.

Stereotactic injection of the therapeutic virus into the nervous system as described by During et al., Science 266, 1399-403 (1994) is an accepted, efficient and widely used procedure for introducing substances to, or biopsying from, specific regions of the CNS in both humans and animals.

A further method according to the present invention includes administration of a Herpes Simplex Virus replication defective vector as disclosed. For example, the replication defective mutant herpes simplex virus is administered subcutaneously, intranasally, intratracheally, or intramuscularly. Administration is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors.

A composition in accord with the present invention can be administered alone or in combination with other treatments, either simultaneously or sequentially dependent upon the condition to be treated.

Pharmaceutical compositions according to the present invention, and for use in accordance with the present invention, can comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration which, for instance, can be oral, or by injection, e.g., cutaneous, subcutaneous or intravenous.

For intravascular, cutaneous, subcutaneous, intramuscular, intraocular or intracranial injection, or direct injection into cerebrospinal fluid, injection into the biliary tree, or injection at the site of affliction, the active ingredient preferably will be in the form of a parenterally acceptable aqueous solution which is pyrogen-free and has suitable pH, isotonicity and stability. Those of skill in the relevant art are well able to prepare suitable solutions using, for example, isotonic vehicles such as Sodium Chloride Injection, Ringer's Injection, Lactated Ringer's Injection and like components well known to those in the pharmaceutical sciences, e.g., Remington's Pharmaceutical Science, latest edition (Mack Publishing Company, Easton, Pa.). Preservatives, stabilizers, buffers, antioxidants and/or other additives may be included, as required.

HSV replication defective vectors and cells containing them are not just useful in a therapeutic context. It is generally useful to be able to express nucleic acid stably within a host cell in vitro. The results reported herein indicate that stable, long-term expression can be achieved using constructs of the invention. Therefore, this can be employed in the production of a product encoded by a nucleotide sequence of interest, heterologous to the U_{L}29 region of a herpes virus, which can if need be recovered from a host cell for subsequent use. Polypeptides, for instance, are useful in raising antibodies in animals which can be used in the generation of hybridomas for monoclonal antibody production, and selection of antibodies or other binding molecules on columns or by means of bacteriophage display. A polypeptide produced by expression from a heterologous nucleotide sequence included in a construct according to the present invention may, of course, have a multitude of other uses, depending on the nature of the polypeptide itself.

In another preferred embodiment, heterologous nucleic acid sequences, fragments and derivatives thereof encode polypeptides of a sufficient length such that they activate the immune system resulting in the lysing of infected cells, such as, for example cells infected with Hepatitis B virus. Preferred genes, fragments or derivatives thereof, include those that have at least about 70 percent homology (sequence identity) to a wild type gene, more preferably about 80 percent or more homology to wild type gene, still more preferably about 85 to 90 percent or more homology to wild type gene. Examples of wild type genes are shown in Table 2.

To determine the percent identity or homology of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The nucleotides at corresponding nucleotide positions are then compared. When a position in the first sequence is occupied by the nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two nucleotide sequences is determined using the GAP program in the GCG software package (available at online through the Genetics Computer Group), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of Meyers and Miller (Comput. Appl. Biosci. 4:11-17 (1988)) which has been incorporated into the ALIGN program (version 2.0 or version 2.0U), using a PAM120 weight residue table, a gap length penalty of 12 and a gap penalty of 4.

Examples of viral organisms include, but are not restricted to, those listed in table 1. For information about the viral organisms see Fields of Virology, 3. ed., vol 1 and 2, B N Fields et al. (eds.). Non-limiting examples of genes of selected viral organisms are listed in table 2.

TABLE 1
Selected viral organisms causing human diseases.

| | |
|---|---|
| Herpesviruses | Alpha-herpesviruses: |
| | Herpes simplex virus 1 (HSV-1) |
| | Herpes simplex virus 2 (HSV-2) |
| | Varicella Zoster virus (VZV) |
| | Beta-herpesviruses: |
| | Cytomegalovirus (CMV) |
| | Herpes virus 6 (HHV-6) |
| | Gamma-herpesviruses: |
| | Epstein-Barr virus (EBV) |
| | Herpes virus 8 (HHV-8) |
| Hepatitis viruses | Hepatitis A virus |
| | Hepatitis B virus |
| | Hepatitis C virus (see Example 4) |
| | Hepatitis D virus |
| | Hepatitis E virus |
| Retroviruses | Human Immunodeficiency 1 (HIV-1)(see |
| | Example 3) |
| Orthomyxoviruses | Influenzaviruses A, B and C |
| Paramyxoviruses | Respiratory Syncytial virus (RSV) |
| | Parainfluenza viruses (PI) |
| | Mumps virus |
| | Measles virus |
| Togaviruses | Rubella virus |
| Picornaviruses | Enteroviruses |
| | Rhinoviruses |
| | Coronaviruses |
| Papovaviruses | Human papilloma viruses (HPV) |
| | Polyomaviruses (BKV and JCV) |
| Gastroenteritisviruses | |
| Filoviridae | |
| Bunyaviridae | |
| Rhabdoviridae | |
| Flaviviridae | |

TABLE 2
Genes of viral organisms

| | | Open | |
|---|---|---|---|
| | | reading | |
| Organism | Target gene | frame | Gene product |
| HIV | gag: | MA | p17 |
| | | CA | p24 |
| | | NC | p7 |
| | | | p6 |
| | pol: | PR | p15 |
| | | RT | p66 |
| | | | p31 |
| | env: | | gp120 |
| | | | gp41 |
| | tat | | transcriptional transactivator |
| | rev | | regulator of viral expression |
| | vif | | |
| | vpr | | |
| | vpu | | |
| | nef | | |
| RSV | NS1 | | |
| | NS2 | | |
| | L | | |
| | 2-5A-dependent | | |
| | Rnase L | | |
| HPV | E1 | | helicase |
| | E2 | | transcription regulator |
| | E3 | | |
| | E4 | | late NS protein |
| | E5 | | transforming protein |
| | E6 | | transforming protein |
| | E7 | | transforming protein |
| | E8 | | |
| | L1 | | major capsid protein |
| | L2 | | minor capsid protein |
| HCV | NS3 | | protease |
| | NS3 | | helicase |
| | HCV-IRES | | (see Example 4) |
| | NS5B | | polymerase |
| HCMV | DNA polymerase | | |
| | IE1 | | |
| | IE2 | | |
| | UL36 | | |
| | UL37 | | |
| | UL44 | | polymerase asc. protein |
| | UL54 | | polymerase |
| | UL57 | | DNA binding protein |
| | UL70 | | primase |
| | UL102 | | primase asc. protein |
| | UL112 | | |
| | UL113 | | |
| | IRS1 | | |
| VZV | | 6 | |
| | | 16 | |
| | | 18 | |
| | | 19 | |
| | | 28 | |
| | | 29 | |
| | | 31 | |
| | | 39 | |
| | | 42 | |
| | | 45 | |
| | | 47 | |
| | | 51 | |
| | | 52 | |
| | | 55 | |
| | | 62 | |
| | | 71 | |
| HSV | IE4 | | US1 |
| | IE5 | | US12 |
| | IE110 | | ICP0 |
| | IE175 | | ICP4 |
| | UL5 | | helicase |
| | UL8 | | helicase |
| | UL13 | | capsid protein |
| | UL30 | | polymerase |
| | UL39 | | ICP6 |
| | UL42 | | DNA binding protein |

Information about the above selected genes, open reading frames and gene products is found in the following references: Field A. K. and Biron, K. K. “The end of innocence” revisited: resistance of herpesviruses to antiviral drugs. Clin. Microbiol. Rev. 1994; 7: 1-13. Anonymous. “Drug resistance in cytomegalovirus: current knowledge and implications for patient management”. J. Acquir. Immune Defic. Syndr. Hum. Retrovir. 1996; 12: S1-SS22. Kelley R et al. Varicella in children with perinatally acquired human immunodeficiency virus infection. J Pediatr 1994; 124: 271-273. Hanecak et al. “Antisense oligonucleotides inhibition of hepatitis C virus gene expression in transformed hepatocytes.” J Virol 1996; 70: 5203-12. Walker Drug discovery Today 1999; 4: 518-529. Zhang et al. “Antisense oligonucleotides inhibition of hepatitis C virus (HCV) gene expression in livers of mice infected with an HCV-Vaccinia virus recombinant.” Antim. Agents Chemotherapy 1999; 43, 347-53. Feigin R D, Cherry J D, eds. Textbook of pediatric infectious diseases. Philadelphia: W B Saunders, 1981. Chen B. et al., “Induction of apoptosis of human cervical carcinoma cell line SiHa by antisense oligonucleotide og human papillomavirus type 16 E6 gene.” 2000; 21(3): 335-339. The human herpesviruses. New York: Raven Press; 1993. DeClerque E, Walker R T, eds. Antiviral drug development: a multi-disciplinary approach. Plenum; 1987. Antiviral Drug Resistance (Richman, D. D., ed.), Wiley, Chichester, 1995. Flint S J et al. eds. Principles of virology: Molecular biology, Pathogenesis and Control.

It should be appreciated that, in the above table 2, an indicated gene means the gene and all currently known variants thereof, including the different mRNA transcripts that the gene and its variants can give rise to, and any further gene variants which may be elucidated. In general, however, such variants will have significant sequence identity to a sequence of table 2 above, e.g. a variant will have at least about 70 percent sequence identity to a sequence of the above table 2, more typically at least about 75, 80, 85, 90, 95, 97, 98 or 99 percent sequence identity to a sequence of the above table 2. Sequence identity of a variant can be determined by any of a number of standard techniques such as a BLAST program http://www.ncbi.nlm.nih.gov/blast/). Sequences for the genes listed in Table 2 can be found in GenBank (http://www.ncbi.nlm.nih.gov/). The gene sequences may be genomic, cDNA or mRNA sequences. Preferred sequences are viral genes containing the complete coding region and 5′ untranslated sequences that are involved in viral replication.

As mentioned, a most preferred embodiment the replication defective herpes simplex virus encodes a heterologous gene which codes for an antigenic epitope useful in the treatment of a disease caused by an infectious agent. However, the replication defective mutant HSV, according to the present invention, has many practical therapeutic applications in diseases other than those caused by infectious disease agents.

HSV establishes a natural latent state in sensory ganglia neurons that can last the life time of the individual. There is also experimental evidence that defective HSV mutants, which have had certain of the genes required for a productive acute infection deleted, can also establish latency in CNS neurons.

There are many potential gene therapy applications within the nervous system. One of the main aims is to be able to complement various inborn errors of metabolism which affect the CNS by delivery of a copy of the missing gene. For many such conditions (e.g. Gaucher's Disease) it has been shown that the metabolic defects can be corrected by providing pharmacological amounts of the missing protein. Providing large quantities of such enzymes for pharmacotherapy is often not possible, and, where it is possible, extremely expensive. However, delivery using the present invention of the missing gene directly to the nervous system cells with long-term expression of physiological levels of protein, provides for correction of the metabolic defect with a single treatment. Thus, the present invention may be used for treating a large number of such inherited conditions such as neuronopathic Gaucher's disease and other lysosomal storage disorders, metachromatic leucodystrophy, G_{M2} gangliosidosis, Huntingdon's disease and so on.

There are other neurological diseases due to acquired metabolic abnormalities. In Parkinson's disease there is a loss of cells in the substantia nigra which leads to a deficiency of the neurotransmitter dopamine in the caudate nucleus. Patients can show a good therapeutic response to dopaminergic drugs, but if given orally large doses are needed and the side effects often limit treatment. In this condition it has been shown that providing a local source of the missing chemical in the basal ganglia of the brain can lead to dramatic resolution of symptoms. By delivering the tyrosine hydroxylase gene (which codes the enzyme that makes dopamine) to the cells of the basal ganglia, it is possible to obtain symptomatic relief in experimental models of the disease. Again, the present invention provides a way of obtaining long-term gene expression which may be useful in any of these applications. The approach can be usable for a number of other degenerative diseases of the nervous system, such as some of the dementias.

Gene therapy also has applications in the treatment of cancer. There are a few rare cancers and neoplastic syndromes that are due to inherited genetic defects (i.e. retinoblastoma, multiple endocrine neoplasia, neurofibromatosis). Some of these may be treated or even prevented by supplying the missing gene by gene therapy. A number of these diseases can affect the cells of the nervous system. Again Herpes Simplex Virus replication defective vector according to the invention can be useful in treatments.

All cancer involves the accumulation of a number of acquired genetic abnormalities which combine to lead to uncontrolled cell division and growth. As more is learned about neoplasia, it is becoming clear that there are a small number of genes which are very important in the aetiology of a large number of cancers. Tumor suppressor genes, such as p53, are good examples of genes which have become inactivated in many neoplastic cell types. Gene therapy can be used to introduce a functional copy of the mutated gene into the neoplastic cells and, hence, to arrest their growth. Herpes Simplex Virus replication defective vectors as disclosed herein can be useful for such applications within the nervous system.

Another approach to gene therapy for cancer is to deliver a suicide gene specifically to the malignant cells which would produce a product which could, directly or indirectly, lead to their death. An example of such a strategy is to use the HSV thymidine kinase (TK) gene containing Herpes Simplex Virus replication defective vectors to specifically transduce malignant cells. The patient is then given acyclovir, which is only metabolized to its active, toxic, metabolite in cells which contain the HSV TK gene. With specific delivery of such a suicide gene to neoplastic cells, constructs of the invention can be used to produce gene expression in neural derived tissue.

### EXAMPLES

Materials and Methods

Cells and Viruses.

Vero cells were purchased from the American Type Culture Collection (Manassas, Va.) and used for propagation of wild-type virus strains. The Vero-derived cell line V8-27 was used for propagation of replication-defective mutant viruses. V8-27 cells express the viral proteins ICP8 and ICP27 upon HSV infection. Cells were grown in Dulbecco's modified Eagle's medium (DMEM; Invitrogen, Carlsbad, Calif.) supplemented with 5% bovine calf serum, 5% fetal calf serum (HyClone, Logan, Utah), L-glutamine, penicillin G, and streptomycin (Glu-Pen-Strep; Irving Scientific, Santa Ana, Calif.) at 37° C. with 5% CO₂. Following viral infection, cells were maintained in DMEM containing 1% fetal bovine serum (DMEM-V) at 34° C. until harvest.

The replication-defective HSV-1 recombinants and the parental wild-type KOS 1.1 strain were used. The d102 and d301 viruses contain large deletions within the U_{L}29 gene. This gene encodes the ICP8 protein, which is important for viral DNA replication. The HD-2 virus expresses the E. coli β-galactosidase protein fused in frame to the N terminus of ICP8 and has been used as an HSV-1 β-galactosidase vector.

Stocks of the viruses were generated as either infected-cell lysates, using methods typical to those of skill in the art except that cell pellets were resuspended in DMEM-V containing 20% glycerol, or cell-free virus stocks. For cell-free virus stocks, HD-2 virions were harvested from the supernatant of infected cells as follows. Infected cells and cellular debris were pelleted by low-speed centrifugation (1,000×g) for 10 min at 25° C. Virus particles were collected from the clarified supernatant by high-speed centrifugation (30,000×g) for 1 h at 4° C. The resulting virus pellet was resuspended in fresh DMEM-V containing 20% glycerol. All viruses were titered on the appropriate complementing cell line using DMEM-V containing 0.1% human immune globulin (Massachusetts Public Health Biologic Laboratories, Boston, Mass.) as the overlay medium.

UV Inactivation of HD-2 Virus.

HD-2 virus was partially inactivated by exposure to 254-nm UV light for 10 min at a distance of 5 cm. Subsequent titering revealed that UV treatment reduced viral infectivity approximately 2,000-fold, from 2×10⁹ PFU/ml to 1×10⁶ PFU/ml.

Mice and Inoculations.

Six-week-old female BALB/cJ mice were purchased from Jackson Laboratories (Bar Harbor, Me.) and acclimated for 1 week prior to use. Groups of mice were immunized once by subcutaneous (s.c.) inoculation into the right flank with 2×10⁶ PFU of KOS1.1, d301, or d102 virus, as indicated, or mock-infected by s.c. inoculation with uninfected Vero cell lysate. In some cases, a second identical inoculation was given 3 weeks later to boost host immunity. Four weeks after the final immunization, mice were inoculated s.c. in the left flank with 2×10⁶ PFU of the HD-2 virus, followed by one or more booster inoculations with HD-2 at 3- or 4-week intervals. Inoculations consisted of virus stock diluted into a volume of 20 μl of sterile, endotoxin-free sodium chloride solution, 0.9% (Sigma, St. Louis, Mo.) per mouse. Mice were housed in accordance with National Institutes of Health (NIH) and Harvard University guidelines.

Serum Collection, ELISA, and Antibody Neutralization.

Blood was collected from the tail vein of each mouse before immunization and at the times indicated during each experiment. Serum was prepared using Becton Dickinson Microtainer serum separators (VWR) and then stored at −20° C. prior to analysis.

Enzyme-linked immunosorbent assays (ELISAs) to determine antigen-specific IgG titers were used. Briefly, 96-well Nunc Maxisorp microtiter plates (VWR) were coated with HSV-1 antigen (Advanced Biotechnologies Inc., Columbia, Md.) at 50 ng per well or with β-galactosidase antigen (Sigma) at 250 ng per well in 50 μl of sodium bicarbonate buffer (pH 9.6) (Sigma) overnight at 4° C. Plates were blocked with phosphate-buffered saline (PBS, pH 7.4) containing 5% (wt/vol) nonfat milk for 1 h at 37° C. and washed with PBS containing 0.05% Tween 20 (PBS/T) using a Skatron CellWasher 600 (Molecular Devices, Sunnyvale, Calif.). Serial twofold dilutions of mouse serum (from 1:100 to 1:12,800) in PBS/T were added and incubated for 2 h at 37° C. Following serum antibody binding, plates were washed with PBS/T and then incubated with a rabbit anti-mouse IgG secondary antibody conjugated to alkaline phosphatase (1:1,000 dilution; Sigma) for 1 h at 37° C. Plates were washed with PBS/T and developed by incubation with the alkaline phosphatase substrate p-nitrophenyl phosphate (Sigma) for 20 min at room temperature, and results were read at 405 nm on a VersaMax microplate reader (Molecular Devices, Sunnyvale, Calif.).

The IgG antibody titer indicated is the mean reciprocal log₂ value of the last dilution resulting in an optical density (OD) reading 0.2 units above that of a control serum (background). In each case, negative OD readings at the 1:100 dilution were scored as positive at a 1:50 dilution (reciprocal dilution 50=log₂ 5.65), and this value (5.65) was used as the limit of detection and subtracted from all results.

Neutralizing antibody titers were determined by plaque reduction in the presence of complement using twofold dilutions of pooled serum from at least five mice. Values reported are the reciprocal dilution at which at least 50% plaque reduction of HSV-1 strain KOS 1.1 was observed.

β-Galactosidase Enzyme Assays.

β-galactosidase enzymatic activity present in the virus stocks was determined using the β-galactosidase enzyme assay system (Promega, Madison, Wis.) according to the manufacturer's recommendations. Protein concentrations were calculated by comparison to a standard curve of freshly prepared β-galactosidase protein (Sigma) on the basis of OD₄₁₀ values.

Cellular Proliferation Assay.

Cellular proliferation responses were measured using cellular proliferation assays. Single-cell suspensions were prepared from whole spleens in complete DMEM. Erythrocytes were lysed using the whole blood erythrocyte lysing kit (R&D Systems, Minneapolis, Minn.), and B cells were removed using Dynabeads Pan B (B220) magnetic beads (Dynal Inc., Lake Success, N.Y.). Lymphocytes were counted, and 2×10⁵ cells were plated in quadruplicate wells of a flat-bottomed 96-well plate (Costar, Cambridge, Mass.) in the presence of soluble HSV-1 (Advanced Biotechnologies Inc.) or β-galactosidase (Sigma) (at 5 μg/well final concentration) or in DMEM alone to a final volume of 200 μl. After incubation for 4 days at 37 C, cells were labeled for 6 h with bromodeoxyunridine (BrdU). BrdU incorporation was measured using an ELISA-based assay (Cell Proliferation ELISA; Roche, Indianapolis, Ind.) using the accompanying protocol and reagents. Results are shown as a fold induction of cell proliferation, which was determined by dividing the signal obtained in the presence of HSV or β-galactosidase antigen by that observed with medium alone.

### Example 1

### Efficacy of HSV-Derived Vectors in the Presence of Preexisting Host Immunity

A mouse model of HSV infection was used with several recombinant viruses to test the efficacy of HSV-derived vectors in the presence of preexisting host immunity. The HSV-1 recombinants used are shown in FIG. 1. In most experiments, groups of BALB/cJ mice were immunized by s.c. inoculation with 2×10⁶ PFU of a replication-defective HSV-1, either d301 or d102. The immunization protocol was a single s.c. inoculation with the replication-defective HSV-1 mutant d301 as this protocol: protects mice from death following corneal challenge infection with a highly virulent HSV-1 strain; significantly reduced shedding of challenge virus from the eye; significantly enhanced the rate of virus clearance from the eye following challenge; markedly diminished the establishment of latent infection by this challenge virus. By any of these measures, immunization with d301 was shown to be equivalent to immunization with its replication-competent parental virus KOSL1. In addition, immunization of mice with either d301 or KOS1.1 induced similar neutralizing antibody and T-cell proliferative responses and comparable primary and memory CTL responses.

### Example 2

### Immunity Generated by a Replication-Competent Virus

To directly compare immunity generated by a replication-competent virus, in one experiment a group of mice was immunized with the parental, replication-competent HSV-1 strain KOS 1.1. Four weeks after immunization, mice were inoculated s.c. with 2×10⁶ PFU of the replication-defective HSV-1 mutant HD-2 in the opposite flank. The HD-2 mutant expresses β-galactosidase fused to the N-terminal portion of the viral ICP8 protein and has been used as a model HSV-derived vector (β-galactosidase vector). B-cell responses were assayed by measuring the total IgG antibody responses directed towards HSV and β-galactosidase by ELISA at various times following immunization, primary β-galactosidase vector inoculation, and secondary β-galactosidase vector inoculation. In one experiment, cellular proliferative responses against HSV and β-galactosidase were determined to measure the induction of cellular immunity. By comparing the β-galactosidase-specific antibody responses between virus-inoculated and mock-infected animals, the relative efficacy of the HSV vector in immune and naïve mice, was determined.

### Example 3

### IgG Antibody Responses in Mice to the Infectious Dose of Replication-Defective Virus Vector

To establish that this system provided a quantitative measure of host immunity, a determination of whether the humoral immune response generated by the β-galactosidase vector was proportional to the dose of virus inoculated. The results are shown in FIG. 2. Groups of six mice were inoculated s.c. in the flank with the indicated doses of the HSV-1 β-galactosidase-expressing recombinant HD-2, ranging from 0 (mock infection) to 10 PFU. Three weeks later, mice received an identical booster inoculation with HD-2. Serum samples were collected from each animal prior to infection, at 3 weeks following primary inoculation (primary), and at 3 weeks following secondary inoculation (secondary).

Anti-HSV and anti β-galactosidase IgG titers were determined by ELISA (FIGS. 2A and 2B). Primary and secondary IgG titers to both HSV (FIG. 2A) and β-galactosidase (FIG. 2B) antigens were proportional to the dose of HD-2 virus. These results ensure that the vector inoculum had not surpassed a plateau for measuring increasing IgG antibody responses and that the IgG antibody response to the dose of β-galactosidase vector (2×10⁶ PFU) used in subsequent experiments was within the linear range detected by ELISA.

In addition, one group of mice was inoculated with an equivalent volume of the highest-titer HD-2 virus that had been partially inactivated by treatment with UV light. This exposure resulted in a 2,000-fold reduction in the infectivity of the inoculum (from 1×10⁸ PFU/mouse to 5×10⁴ PFU/mouse). Following primary and secondary infection with UV-treated HD-2, a reduction, was observed, in the IgG antibody responses against both HSV-1 (FIG. 2A; UV primary and UV secondary) and β-galactosidase (FIG. 2B; UV primary and UV secondary) compared to the untreated HD-2 inoculum (10⁸ PFU/mouse). Furthermore, the antibody response was proportional to the viral PFU and not to the amount of input protein, because the IgG antibody titers elicited by UV-treated HD-2 were similar to IgG titers resulting from untreated HD-2 inoculated at 10⁴ PFU/mouse. These results indicated that the antibody response was mainly a result of viral infection and was not simply due to input viral or β-galactosidase antigen.

### Example 4

### Prior HSV Immunity and Induction of a Durable β-Galactosidase Antibody Response by an HSV Vector

To assess the effect of prior HSV-1 infection on the ability of an HSV-1-derived vector to generate IgG antibody responses, mice were immunized once with 2×10⁶ PFU of a replication-defective HSV-1 mutant containing a deletion within the U_{L}29 gene encoding the ICP8 protein, either d301 or d102, or mock-infected animals with uninfected Vero cell lysate, as indicated. To verify immunization following a single d301 inoculation, anti-HSV neutralizing antibody was measured in the serum of these mice, and a neutralization titer of 64 was obtained. This value was similar to previous results with d301 in mice and comparable to neutralizing titers observed in humans (range, 12 to 372).

At 4 and 8 weeks after the immunization, all mice were inoculated with 2×10⁶ PFU of the β-galactosidase vector HD-2. Serum samples were collected from the mice, and HSV-1-specific and β-galactosidase-specific IgG antibody titers were measured by ELISA (FIG. 3). Despite the induction of an antiviral antibody response following inoculation with d301 or d102 (FIG. 3A, week 4), indicative of preexisting immunity, the generation of β-galactosidase-specific antibody was equivalent between the immune groups and mock controls following HD-2 inoculation (FIG. 3B, weeks 4 to 12).

### Example 5

### Persistence of a Stable Antiviral Neutralizing Antibody in Experimentally Infected Mice

Replication-competent and replication-defective HSV strains are capable of eliciting a stable antiviral neutralizing antibody response in experimentally infected mice that persists for at least 7 months post-infection. To further address this issue, the ability of HD-2 to elicit durable IgG antibody responses following inoculation in these immune mice was evaluated. Serum from the HD-2 inoculated mice was collected up to one year following vector administration and boost, and the HSV-1-specific and β-galactosidase-specific IgG antibody titers were determined. The results showed that inoculation with HD-2 generated a remarkably durable antibody response toward HSV-1 (FIG. 3C, weeks 15 to 60) and β-galactosidase (FIG. 3D, weeks 15 to 60) antigens and that prior HSV immunity did not alter the longevity of this antibody response. These results showed that prior immunity to HSV-1 did not diminish the ability of an HSV-1-derived vector to generate durable, high-titer antibody responses.

### Example 6

### Effectiveness of HSV Vectors in the Presence of Boosted Antiviral Immunity

Because recurrent HSV infection may increase the host response directed against the virus, the antiviral immunity was enhanced to address vector efficacy. Mice were immunized with 2×10⁶ PFU of d301 and to boost the response, the mice were immunized with the same dose of d301 three weeks later or mock-infected mice with uninfected Vero cell lysate, at both times. The addition of a d301 boost resulted in an observed neutralizing antibody titer of 1,024 and an increase in the anti-HSV IgG titer (FIG. 4A, weeks 3 and 7). At 4, 7, and 10 weeks after the second immunization, mice were inoculated with 2×10⁶ PFU of HD-2. Despite the increased anti-HSV response elicited with the immunization-boost regimen, equivalent β-galactosidase IgG titers were generated by HD-2 inoculation in mock-infected and immunized groups of animals (FIG. 4B, weeks 7 to 18). This result indicated that even a heightened level of HSV immunity was unable to reduce the capacity of an HSV vector to generate IgG antibody responses.

### Example 7

### Antibody Response Induction by a Purified HSV Vector in Immune Mice

To confirm that the observed β-galactosidase-specific antibody responses were the result of vector-expressed antigen rather than protein present in the inoculum, cell-free stocks of the HD-2 virus from the supernatant of infected cells was prepared, as described in Materials and Methods, supra. β-galactosidase enzyme activity was measured for virus stocks, and cell-free stocks were found to contain 65-fold less β-galactosidase activity than virus stocks prepared as infected-cell lysates.

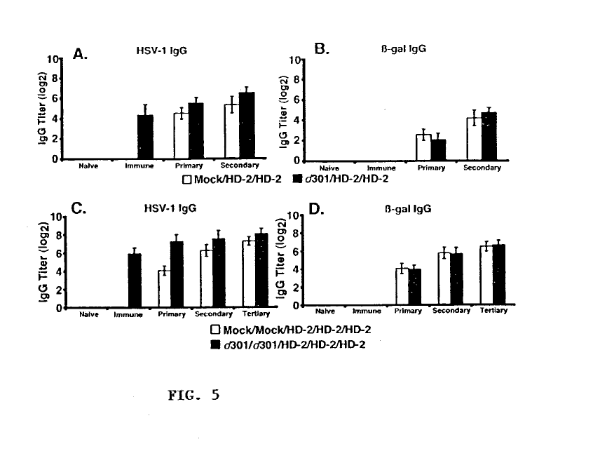

The effect of a single immunization with d301 on the efficacy of the cell-free HD-2 virus stock to elicit a β-galactosidase-specific IgG antibody response (FIGS. 5A and B), was determined. Groups of mice were immunized once at week 0 by s.c. inoculation with 2×10⁶ PFU of d301 or mock infected. At weeks 4 and 7, all mice were inoculated s.c. with 2×10⁶ PFU of the cell-free HD-2 virus. Serum samples were collected at the beginning of the experiment (naive), at week 3 (immune), at week 7 (primary), and at week 9 (secondary). Despite the generation of an HSV-specific immune response following a single d301 inoculation (FIG. 5A, immune), cell-free HD-2 virus elicited a β-galactosidase-specific IgG response (FIG. 5B, primary and secondary) that was similar in magnitude to previous results obtained with preparations of virus in infected-cell lysate (FIG. 3B).

In a second experiment, the effects of two d301 immunizations on the cell-free HD-2 virus was assessed (FIGS. 5C and 5D). Two groups of six mice were immunized by s.c. inoculation with 2×10⁶ PFU of d301 or mock infected at weeks 0 and 3. At weeks 7, 10, and 13, all mice received 2×10⁶ PFU of the cell-free HD-2 virus s.c. in the opposite flank. Naive serum was collected from each mouse prior to week 0, HSV immune serum was collected at week 6, and primary, secondary, and tertiary serum samples were collected at weeks 10, 12, and 15, respectively. Despite the increased level of anti-HSV immunity elicited by the second d301 inoculation (FIG. 5C, immune), the cell-free HD-2 virus generated β-galactosidase-specific IgG antibody responses that were equivalent between mock-treated and immune animals (FIG. 5D, primary, secondary, and tertiary).

The results show that cell-free HD-2 recombinant virus elicited β-galactosidase-specific antibody responses in mice immunized either once or twice with d301 that were equivalent to those of mock-infected controls. These results also reiterate the point that the humoral responses generated by HD-2 are due to vector infection and not input antigen. The IgG response to UV-treated virus is PFU dependent (FIGS. 2A and 2B, UV-treated viruses) and the antibody response is similar when either infected-cell lysate (FIGS. 3A-3D and FIGS. 4A and 4B) or partially purified virions (FIGS. 5A-5D) are used for inoculation.

### Example 8

### Immunity Generated with Replication-Competent HSV-1

In each of the previous experiments, replication-defective viruses were used to generate antiviral immunity. The experiments were designed in this way because immunization with a replication-defective virus is as effective as immunization with a replication-competent virus for inducing protective immunity and the immune phenotype of d301-immunized mice is comparable to that of KOS 1.1-immunized animals. To determine if there were any qualitatively differences in immunity generated by a replication-competent virus from that elicited by a replication-defective virus in this model, prior infection was addressed with a replication-competent virus was able to inhibit an HSV vector.

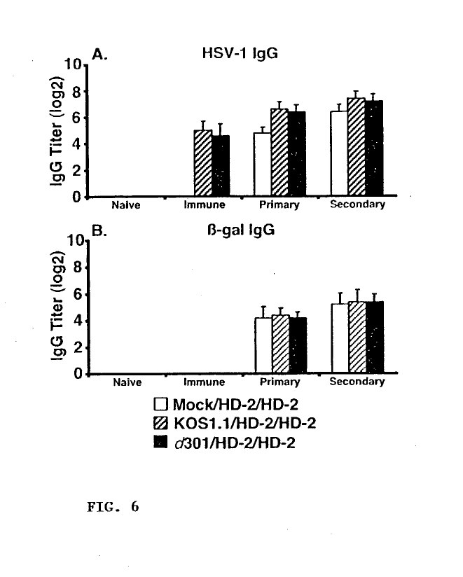

The efficacy of the cell-free HD-2 vector in mice inoculated with either the replication-defective virus d301 or the replication-competent parental virus KOS1.1 (FIGS. 6A-6B) was compared. Groups of mice were immunized by s.c. inoculation in the right flank with 2×10⁶ PFU of either KOS1.1 or d301 or mock treated. A neutralizing antibody titer of 128 was measured in the KOS 1.1-immunized mice, a value similar to that observed following a single d301 inoculation in mice and values reported for human serum. At weeks 4 and 7, all mice were inoculated s.c. with 2×10⁶ PFU of cell-free HD-2 virus. Serum samples were collected before immunization (native), at week 3 (immune), at week 7 (primary), and at week 10 (secondary). Despite phenotypic differences in their ability to replicate in vitro and in vivo, KOS1.1 and d301 viruses generated equivalent antiviral IgG responses (FIG. 6A, immune) compared to mock-infected animals. Despite preexisting antiviral immunity following KOS 1.1 or d301 infection, HD-2 was able to elicit equivalent β-galactosidase-specific antibody responses in both groups of mice (FIG. 6B, primary and secondary). Thus, anti-HSV immunity induced by either replication-competent or replication-defective HSV strains did not diminish the vaccine efficacy of the HSV vector.

### Example 9

### Cellular Proliferative Responses to β-Galactosidase

To confirm that β-galactosidase-specific cellular responses were being activated in these mice following vector inoculation, cellular proliferation responses to HSV and β-galactosidase antigens were analyzed. Mice were immunized with a single inoculation of 2×10⁶ PFU of d301 or mock infected. At weeks 4 and 7, all mice were inoculated with 2×10⁶ PFU of cell-free HD-2. Spleens were harvested at week 10 from two mice per group, and lymphocytes were isolated, pooled for analysis, and plated at 2×10⁵ cells per well in a microtiter plate. Cells were cultured in the presence of soluble HSV or β-galactosidase (5 μg/well) for 4 days. Proliferation was measured by BrdU incorporation using an ELISA-based chemiluminescence assay.

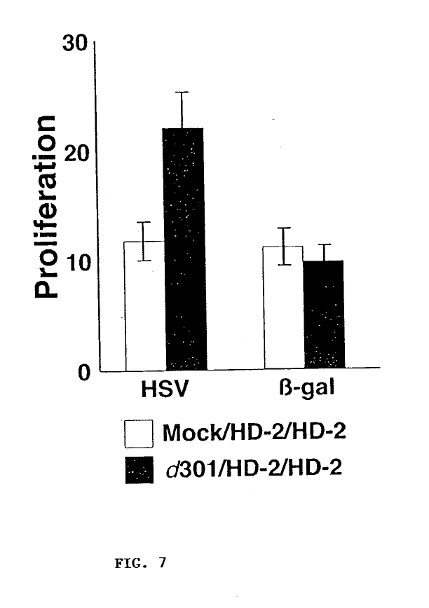

Following immunization and HD-2 inoculation, specific responses were elicited against both HSV and β-galactosidase antigens (FIG. 7). When cultured in the presence of HSV, a 12-fold induction was observed in cells from mock-immunized mice and a 22.5-fold induction was detected in cells from d301-immunized mice compared to cells incubated in medium alone. This difference in cellular activity can be attributed to the fact that the d301-immune mice had received one additional viral inoculation. When cells were cultured in the presence of β-galactosidase, an 11-fold induction was found for mock-immunized mice and a 9.5-fold induction was observed in immune animals. Because this difference was not statistically significant, the proliferative response to β-galactosidase was not diminished by prior d301 immunization. This result indicated that cellular responses to a vector-encoded antigen can also be unaffected in immune mice.

### Example 10

### Clinical

The results from this report are important for testing candidate genital herpes vaccines. We have shown previously, using the same virus (d301), dose (2×10⁶ PFU), and route of inoculation (s.c.), that immunization with a replication-defective HSV protected mice from disease following challenge HSV infection, and always in the absence of sterilizing immunity. The data presented here show that HSV can infect a first round of cells regardless of preexisting host immunity against the virus. In addition, the results show that antibody responses to novel viral antigens can be generated equally well in naïve and HSV-immune hosts.

All of the references identified hereinabove, are hereby expressly incorporated herein by reference to the extent that they describe, set forth, provide a basis for or enable compositions and/or methods which may be important to the practice of one or more embodiments of the present inventions.

The invention has been described in detail with reference to preferred embodiments thereof. However, it will be appreciated that those skilled in the art, upon consideration of this disclosure, may make modifications and improvements within the spirit and scope of the invention.

What is claimed is:

1. A method of inducing in a mammal an immune response against a specific infectious disease agent, the method comprising:
providing a recombinant replication defective mutant Herpes Simplex Virus comprising a herpes simplex virus wherein at least a fragment of the U_{L}29 gene is deleted and, in the site of the deletion, a heterologous sequence encoding an antigen from said specific infectious disease causing agent is inserted;
administering to said mammal an immune response eliciting amount of the recombinant replication defective mutant Herpes Simplex Virus; and
eliciting in the mammal an immune response against the infectious disease causing agent.

2. The method of claim 1, wherein said heterologous sequence comprises a promoter Which directs the expression of DNA encoding an antigen from said specific infectious disease causing agent.

3. The method of claim 1, wherein said heterologous sequence is under the control of any herpes simplex virus promoter.

4. The method of claim 1, further comprising eliciting a durable humoral immune response specific for the antigen encoded by said heterologous sequence.

5. The method of claim 1, further comprising eliciting a durable cellular immune response specific for the antigen encoded by said heterologous sequence.

6. The method of claim 1, further comprising obtaining a durable immune response in the presence of prior host immunity to herpes simplex virus.

7. The method of claim 1, further comprising administering multiple inoculations using the same replication defective herpes simplex virus and continuing to elicit a durable immune response to the antigen encoded by said replication defective mutant herpes simplex virus.

8. The method of claim 1, wherein said replication defective mutant herpes simplex virus comprises sequences encoding one or more antigenic epitopes.

9. The method of claim 1, further comprising administering at least about one other vaccine with said replication defective mutant herpes simplex virus in a series of inoculations.

10. The method of claim 9, further comprising eliciting specific immune responses to the same antigen with said vaccine(s) and said replication defective mutant herpes simplex virus.

11. The method of claim 9, further comprising eliciting an immune response to a different antigenic epitope of the infectious disease causing agent with the vaccine than the antigenic epitope response elicited with said replication defective mutant herpes simplex virus.

12. The method of claim 9, further comprising increasing the durability of immune responses induced by said vaccine(s) by immunizing with said vaccine(s), followed by at least about one booster immunization with said replication defective mutant herpes simplex virus.

13. The method of claim 1, wherein the administering step comprises administering said replication defective mutant herpes simplex virus subcutaneously, intranasally, intratracheally, or intramuscularly.

14. The method of claim 13, wherein the administering step comprises administering said replication defective mutant herpes simplex virus subcutaneously.

15. The method of claim 1, wherein said infectious disease causing agent is selected from the group consisting of a virus, a bacteria, a parasite, a protozoa and a fungus.

16. A vector comprising a replication defective herpes virus vector, wherein the replication defective herpes virus vector comprises a deletion of at least a fragment of a U_{L}29 gene and replacing said deletion with a heterologous sequence encoding an antigen from a specific infectious disease agent, wherein said vector can express said antigen.

17. The vector of claim 16, wherein a fusion protein comprising a fragment of the U_{L}29 gene product and heterologous gene product can be expressed.

18. The vector of claim 17, wherein any portion of the U_{L}29 gene is deleted and replaced by the heterologous sequence.

19. A replication defective recombinant mutant herpes simplex virus vector capable of infecting mammalian cells, the vector comprising at least about one heterologous gene inserted into a region where at least a fragment of a U_{L}29 gene was deleted, the deletion being non essential for viral replication, wherein, either:
(i) the heterologous gene is operably linked to at least a fragment of said U_{L}29 gene; or,
(ii) the heterologous gene is operably linked to a promoter which controls its expression; or,
(iii) the heterologous gene comprises sequences from the U_{L}29 gene which are controlled by a HSV promoter,
whereby a fusion protein comprising the heterologous sequence gene product and at least a portion of a U_{L}29 gene product is expressed.

20. The vector of claim 19, wherein said HSV replication defective vector comprises a heterologous sequence which encodes for, and is capable of, expressing a specific antigen associated with a disease and is capable of eliciting an immune response when administered to a patient in need of treatment.

21. The HSV replication defective vector of claim 19, wherein said HSV replication defective vector is capable of expressing the antigen to induce an immune response in either naïve or pre-immunized animals.

22. The HSV replication defective vector of claim 21, wherein said immune response is humoral.

23. The HSV replication defective vector of claim 21, wherein said immune response is cellular.
